(19) United States
(12) Patent Application Publication
(10) Pub. No.: US 2025/0177726 A1
(43) Pub. Date: Jun. 5, 2025

(54) HEMODYNAMIC SUPPORT SYSTEMS AND METHODS

(71) Applicants: Narwhal Medical LLC, Minneapolis, MN (US); Boston Scientific SciMed, Inc., Maple Grove, MN (US)

(72) Inventors: Loïc Van Horne, Minneapolis, MN (US); Peter T. Keith, St. Paul, MN (US); Qian Liu, Plymouth, MN (US); Benjamin Carl Breidall, Eden Prairie, MN (US)

(21) Appl. No.: 18/529,527

(22) Filed: Dec. 5, 2023

(51) Int. Cl. A61M 60/808; A61M 60/13; A61M 60/17; A61M 60/216; A61M 60/414

(52) U.S. Cl. CPC A61M 60/808; A61M 60/13; A61M 60/17; A61M 60/216; A61M 60/414

(57) ABSTRACT

Some embodiments of percutaneous ventricular assist devices have an expandable pump impeller and an associated flexible drive shaft. The drive shaft can be coupled to a motor located external to the patient. The motor can rotate the drive shaft to spin the pump impeller inside of the pump housing, causing blood to be pumped within the patient. In some embodiments, the pump impeller is inflatable and/or self-expandable. The percutaneous ventricular assist devices with inflatable or self-expandable pump impellers are designed to have very small delivery profiles. Accordingly, various deployment modalities, including radial artery deployment, are practicable using the two-part percutaneous ventricular assist devices described herein.

### TECHNICAL FIELD

The present disclosure relates to mechanical hemodynamic support systems, devices, and methods, such as expandable percutaneous ventricular assist devices with inflatable pump impellers and usage methods.

### BACKGROUND

Mechanical hemodynamic support devices, such as percutaneous ventricular assist devices (“pVADs”) and other devices, are currently used in interventional cardiology to perform protected percutaneous coronary intervention (“PCI”). During such procedures, mechanical hemodynamic support is either placed prophylactically or readily available in the event of a complication. If a complication occurs, having hemodynamic support to continue circulating blood throughout the body while the complication is mitigated provides significant patient benefits.

Additionally, pVADs can be used to offload the heart prior to performing PCI. As a result, the work required by the heart to pump blood is reduced because the pVAD takes on a significant portion of the pumping load. There is clinical evidence showing that offloading the heart during a myocardial infarction has long-term beneficial effects for myocardial tissue viability.

Current pVADs require a relatively large access site to accommodate delivery. These devices are too large to be placed through a radial access site. These large access site requirements require vascular access via large vessels such as the femoral artery, the axillary artery, or in the venous system. Large access sites require longer patient follow-up and are more prone to bleeding complications than a radial access site.

Moreover, large access sites require some form of access site closure device or surgical cut down and suturing post-procedurally while smaller access sites can be managed with pressure alone.

Additionally, the sizes of current pVADs are too large for many patients throughout the world including children and people with smaller body structures. Accordingly, improved systems, devices, and methods would be beneficial.

### SUMMARY

This disclosure describes blood pump systems such as percutaneous ventricular assist devices, and methods for their deployment and use. This disclosure, is further focused specifically on the expandable impeller used for the blood pump as well as the positioning of the impeller within the pump housing.

One example of such a system described herein includes a medical pump that includes an elongate drive shaft defining a lumen, and an impeller attached to a distal end of the drive shaft. The impeller can include an inflatable portion fluidly coupled to the lumen, and a noninflatable web portion attached to and peripherally surrounded by the inflatable portion.

Such a medical pump may optionally include one or more of the following features. The inflatable portion may comprise an oblong torus when inflated. The web portion may be within the oblong torus. The inflatable portion may include a first inflatable blade member extending from the distal end of the drive shaft and a second inflatable blade member extending from the distal end of the drive shaft. Distal end portions of the first and second inflatable blade members may be conjoined at a distal end of the inflatable portion. The first and second inflatable blade members may extend along spiral paths from the distal end of the drive shaft to the distal end of the inflatable portion. The inflatable portion may include three or more inflatable blade members that each extend from the distal end of the drive shaft and that conjoin at a distal end of the inflatable portion. The three or more inflatable blade members may extend along spiral paths from the distal end of the drive shaft to the distal end of the inflatable portion.

In another aspect, this disclosure is directed to a medical pump that includes an elongate drive shaft defining a lumen, and an impeller attached to a distal end portion of the drive shaft. The impeller can include: (i) a first inflatable blade member extending spirally between a proximal end and a distal end of the first inflatable blade member, wherein the proximal and distal ends of the first inflatable blade member are each attached to the distal end portion of the drive shaft; (ii) a second inflatable blade member extending spirally between a proximal end and a distal end of the second inflatable blade member, wherein the proximal end distal ends of the second inflatable blade member are each attached to the distal end portion of the drive shaft; (iii) a first noninflatable web portion attached to the first inflatable blade member and to the distal end portion of the drive shaft; and (iv) a second noninflatable web portion attached to the second inflatable blade member and to the distal end portion of the drive shaft.

Such a medical pump may optionally include one or more of the following features. The impeller may also include a third inflatable blade member extending spirally between a proximal end and a distal end of the third inflatable blade member. The proximal end distal ends of the third inflatable blade member may be each attached to the distal end portion of the drive shaft. A third noninflatable web portion may be attached to the third inflatable blade member and to the distal end portion of the drive shaft. The impeller may be reconfigurable between a deflated low-profile delivery configuration and an inflated operable configuration that is radially expanded in comparison to the low-profile delivery configuration. When the impeller is in the inflated operable configuration, the first and second impeller blades may each spiral around the distal end portion of the drive shaft by at least 30°, or at least 60°, or at least 90°, or at least 120°, or at least 150°, or at least 180°.

In another aspect, this disclosure is directed to a medical pump that includes an inflatable impeller that is rotatable about an axis and that has an operable configuration comprising an inflated conical portion; and multiple blade members extending axially from the conical portion.

Such a medical pump may optionally include one or more of the following features. In some embodiments, the multiple blade members are inflatable, however the multiple blade members are deflated while the inflatable impeller is in the operable configuration. The multiple blade members can consist of three blade members. The medical pump may also include an elongate drive shaft. The conical portion may be attached to a distal end portion of the elongate drive shaft. In some embodiments, the inflatable impeller is configurable in three configurations comprising: the operable configuration; an uninflated low-profile delivery configuration; and a fully inflated configuration. The operable configuration includes the multiple blade members, but in the fully inflated configuration the multiple blade members are not included or defined. In some embodiments, the multiple blade members are inflatable and the multiple blade members are inflated while the inflatable impeller is in the operable configuration. In some such embodiments, the inflatable impeller includes a plurality of noninflatable web portions, and wherein each noninflatable web portion is attached to a respective blade member of the multiple blade members. When the inflatable impeller is in the operable configuration, the multiple blade members may each spiral around the axis by at least 50°. The multiple blade members may comprise three blade members that are each inflatable, and the inflatable impeller may further comprise three noninflatable web portions. Each noninflatable web portion of the three noninflatable web portions may be attached to and located radially within a respective blade member of the three blade members. When the inflatable impeller is in the operable configuration, the three blade members may each spiral around the axis by at least 50°.

Methods for deploying the percutaneous blood flow assist devices described herein to a target location within a patient may optionally include one or more of at least the following features. The pump housing system may be advanced to the target location via the patient's vasculature. Once the pump housing system is delivered to the target location within a patient, the pump impeller and drive shaft may be tracked through the drive shaft housing and into the pump housing while in a radially collapsed configuration. Once the impeller is located within the pump housing, the impeller may be expanded by supplying an inflation medium (e.g., a fluid or gas) to the pump impeller via an inflation lumen of the drive shaft. With the impeller expanded, the drive shaft may be rotated thereby causing the impeller to rotate within the pump housing. Rotation of the pump impeller relative to the pump housing may cause the pump impeller to self-center relative to the pump housing through the creation of a hydrodynamic bearing. Additionally, the rotation of the impeller may create a fluid pressure gradient and drive blood flow through the pump.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 schematically illustrates an example mechanical hemodynamic support system (or pVAD) providing hemodynamic support to a patient, in accordance with some embodiments of the subject matter disclosed herein.

FIG. 2 is a perspective view of an example inflatable pump impeller that is configured in a low-profile delivery configuration.

FIG. 3 is a perspective view of the inflatable pump impeller of FIG. 2 in a fully inflated configuration.

FIG. 4 is a perspective view of the inflatable pump impeller of FIG. 2 in a partially inflated operational configuration.

FIG. 5 is a perspective view of another example inflatable pump impeller in an inflated operational configuration.

FIG. 6 is a perspective view of another example inflatable pump impeller in an inflated operational configuration.

FIG. 7 is a perspective view of another example inflatable pump impeller in an inflated operational configuration.

FIG. 8 is a perspective view of another example inflatable pump impeller in an inflated operational configuration.

FIG. 9 is a perspective view of another example inflatable pump impeller in an inflated operational configuration.

FIG. 10 is a perspective view of another example inflatable pump impeller in an inflated operational configuration.

FIG. 11 is a perspective view of another example inflatable pump impeller in an inflated operational configuration.

FIG. 12 is a perspective view of another example inflatable pump impeller in an inflated operational configuration.

FIG. 13 illustrates an example pVAD in position to provide hemodynamic support to a patient, in accordance with some embodiments.

FIG. 14 illustrates the pVAD of FIG. 13 in a cross-sectional view while the pump impeller is being rotated during operation of the pVAD.

FIG. 15 illustrates the pVAD of FIG. 13 in a cross-sectional view while the pump impeller is not rotating, resulting in the potential for retrograde blood flow through the pVAD.

FIG. 16 illustrates the pVAD in a cross-sectional view while the pump impeller is not rotating and while the pump impeller is positioned farther distally within the pump housing to thereby limit or prevent the potential for retrograde blood flow through the pVAD.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Mechanical hemodynamic support devices, such as percutaneous ventricular assist devices (“pVADs”) according to embodiments of the present disclosure, are capable of unloading or assisting the heart of a patient for a period of time during, for example, a myocardial infarction, cardiogenic shock, a surgical or interventional procedure, and the like.

In some embodiments, the pVAD devices described herein have a sufficiently small profile to facilitate deployment through a sheath placed in a radial artery access site. Some such embodiments of the pVAD devices described herein have an innovative two-part design that allows very small delivery profiles as required by the radial artery access site, for example. The pVAD devices described herein can also be deployed via other vascular access sites such as, but not limited to, the femoral artery, the axillary artery, trans-apical, and the venous system, to provide a few non-limiting examples.

After the device has been advanced to a desired location, or a target location, for example within the heart of a patient, it is radially expanded from its entry profile, also referred to as a low-profile or collapsed configuration, to its functional pumping size, also referred to as an expanded or operable configuration. Devices according to embodiments of the present disclosure can be configured to provide various flow outputs based upon factors such as, but not limited to, size, impeller design, and impeller rotational speed.

One particular aspect of this disclosure pertains to the design and delivery of the impeller and the rotatable drive shaft, which may be the second component delivered of the two-part delivery of the pVAD.

As used herein, the terms “proximal” and “distal” pertain to the orientation of the devices, not the patient. For example, proximal portions of the devices described herein may reside outside of the patient for manipulation by a clinician operator, while distal portions of the devices are residing within the patient.

In some cases, the pump devices described herein are referred to have a blood “pushing” configuration or a blood “pulling” configuration. In this disclosure, “pushing” is used to describe a pumping arrangement by which blood is caused to flow in a direction from proximal to distal. In this disclosure, “pulling” is used to describe a pumping arrangement by which blood is caused to flow in a direction from distal to proximal.

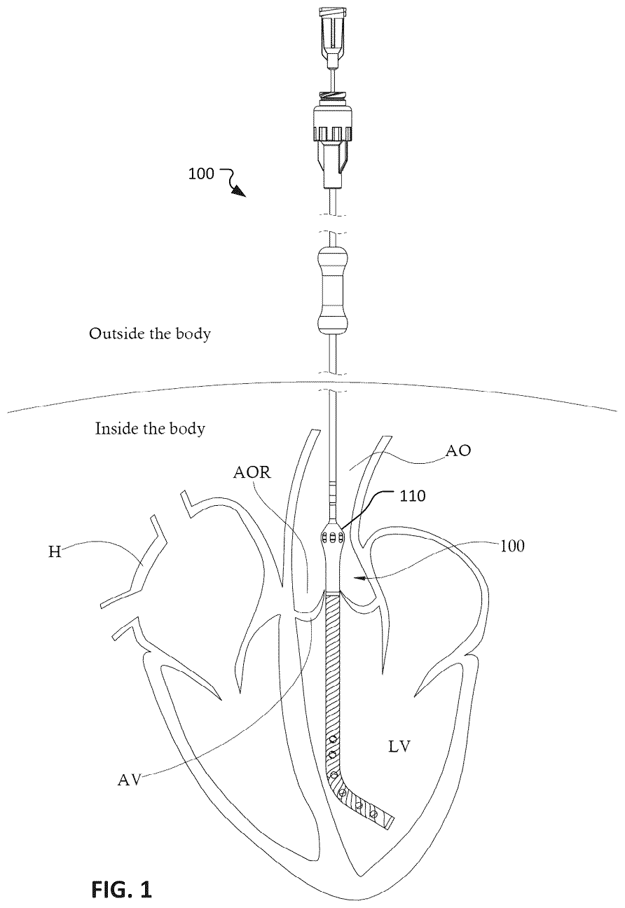

FIG. 1 illustrates an example mechanical hemodynamic support device 100 (or “pump device 100”) percutaneously positioned in the heart H of a patient in a blood pulling configuration, in accordance with some embodiments of the subject matter disclosed herein. The pump device 100 includes a pump housing 110, within which is located an expandable pump impeller (not visible) that is rotatable to thereby pump blood from the left ventricle (“LV”) to the aorta (“AO”). Multiple embodiments of such an expandable pump impeller are described herein.

In the illustrated arrangement, the pump device 100 reaches the heart H by advancing it percutaneously via a vascular access site and through the vasculature of the patient while the pump housing 110 of the pump device 100 is maintained in a low-profile delivery configuration. In the depicted example, once the device is in the desired anatomical location, the pump housing 110 is expanded to its operating size. Then, a separate inner catheter comprising the pump impeller (in a low-profile delivery configuration) and flexible drive shaft is advanced by the clinician into the pump housing 110 to construct or assemble the two-part pump device 100 in vivo.

In the depicted embodiment, a distal tip portion of the pump device 100 is located in the LV. The pump device 100 thereby draws blood out of the ventricle LV and then delivers the blood at a higher pressure to the aortic root (“AOR”) via one or more blood outlets positioned in the AOR. The blood is then circulated throughout the body of the patient by the vasculature of the patient. The pump may also be configured to be placed in any other anatomical location where circulatory support may be needed. Non limiting examples may include placing the pump in the right ventricle, in the descending aorta near the renal arteries, in the carotid arteries, or at the iliac arch.

The motors used to drive the pump impeller of the pump device 100 may be any motor capable of high-speed rotation, such as but not limited to, a brushless motor, a brushed DC motor, or a pneumatically driven turbine.

Further information regarding structures of various embodiments of the pump device 100, and various techniques for deploying the pump device 100, are described in U.S. patent application Ser. No. 18/108,409 filed on Feb. 10, 2023, which is hereby incorporated by reference in its entirety and for all purposes.

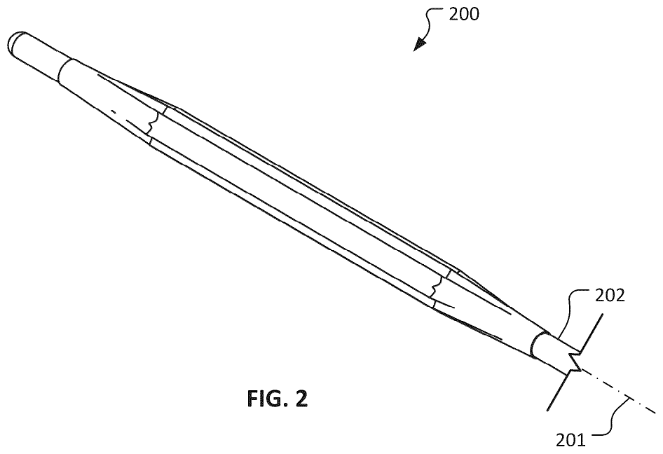

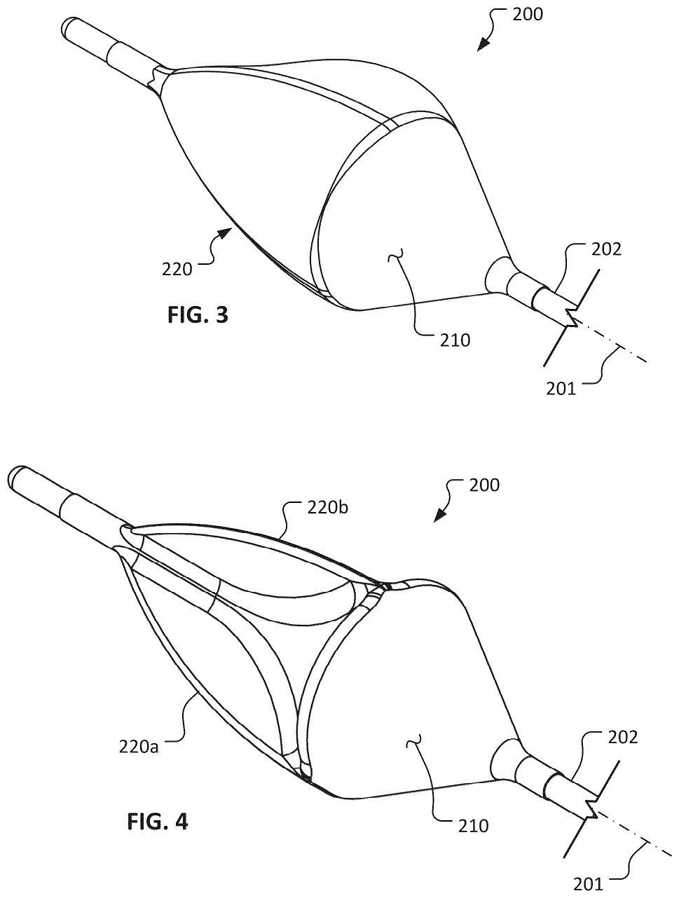

FIGS. 2-4 illustrate an example pump impeller 200 that can be used with a pVAD device such as, but not limited to, the pump device 100. FIG. 2 shows the pump impeller 200 in a deflated low-profile delivery configuration. FIG. 3 shows the pump impeller 200 in a fully inflated configuration. FIG. 4 shows the pump impeller 200 in a partially inflated operable configuration.

The pump impeller 200 is rotatable about an axis 201 to pump blood. The operable configuration (as shown in FIG. 4) includes three blade members 220a, 220b, and 220c (not visible). The blade members 220a-c enable the pump impeller 200 to function as a centrifugal pump as the pump impeller 200 is rotated about the axis 201. In the operable configuration (as shown in FIG. 4) the blade members 220a-c are deflated (not inflated or substantially deflated).

The main purpose of the low-profile delivery configuration of the pump impeller 200 as shown in FIG. 2 is for the delivery (e.g., by transvascular advancement) of the pump impeller 200 into position within the patient in an atraumatic manner. The main purpose of the partially inflated operable configuration of the pump impeller 200 as shown in FIG. 4 is for its ability to pump blood in a normal operational mode. The purpose of the fully inflated configuration of the pump impeller 200 as shown in FIG. 3 is to ensure that the blade members 220 a-c successfully transition from their low-profile delivery configurations (as shown in FIG. 2) to their operable configurations (as shown in FIG. 4). In other words, this intermediate fully inflated configuration (as shown in FIG. 3) may function to assist in un-folding the pump impeller 200 from its low-profile delivery configuration. In the fully inflated configuration (as shown in FIG. 3) the blade members 220a-c are not clearly defined because they are bulging radially outward. Then, when some inflation medium is removed from the pump impeller 200, the blade members 220a-c form (as shown in FIG. 4).

If the in vivo impeller expansion process went directly from the low-profile delivery configuration of the pump impeller 200 (as shown in FIG. 2) to the partially inflated operable configuration of the pump impeller 200 (as shown in FIG. 4) by bypassing the fully inflated configuration of the pump impeller 200 (as shown in FIG. 3), there is a possibility that the pump impeller 200 would not fully unfold/expand or would not properly unfold/expand, thereby resulting in a suboptimal operating profile.

FIG. 4 illustrates the pump impeller 200 partially deflated, which results in the pump impeller 200 having the desired operable profile for pumping blood. Expanding the pump impeller 200 through volumetric expansion (i.e., by filling it with a set volume of fluid and expanding the pump impeller 200 to the fully inflated configuration of FIG. 3) as opposed to pressure expansion (i.e., by filling it to a set pressure) may allow for greater control of achieving the desired operable configuration of the pump impeller 200 (as shown in FIG. 4).

With general reference to inflatable balloon-based pump impellers, when the pump impeller is spinning, it imparts centrifugal force on the inflation medium within the impeller. The impeller will tend to expand in response to the inflation medium's centrifugal force. As a result, additional inflation medium will be drawn into the impeller to fully expand the impeller. With volumetric expansion of the impeller, it may be desired to seal off the proximal end of the inflation lumen once the impeller is inflated. This may prevent the centrifugal force from the spinning impeller to cause the impeller to further expand by preventing additional inflation medium to enter the impeller and over expand the impeller.

As shown in FIG. 4, the pump impeller 200 includes an inflatable conical portion 210 and the multiple blade members 220a-c. In the depicted embodiment, three of the blade members 220a-c are included as part of the pump impeller 200. Alternatively, in some embodiments two blade members, four blade members, or more than four blade members are included as part of the pump impeller 200.

The multiple blade members 220a-c extend axially (along the central axis 201) from the conical portion 210. In the depicted embodiment, the multiple blade members 220a-c extend distally from the conical portion 210. Alternatively, in some embodiments the multiple blade members 220a-c extend proximally from the conical portion 210.

In the depicted embodiment, the pump impeller 200 is attached to a distal end portion of an elongate drive shaft 202. The drive shaft 202 can be rotated by a motor to thereby drive rotations of the pump impeller 200 to pump blood. The drive shaft 202 can define a lumen through which an inflation medium can pass to/from the pump impeller 200.

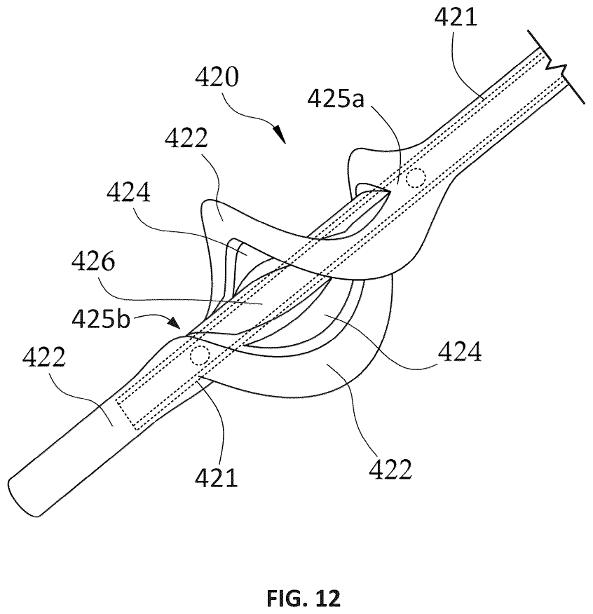

In the depicted embodiment, the multiple blade members 220a-c are not inflated when the pump impeller 200 is in its operable configuration (as shown in FIG. 4). Alternatively, in some embodiments the pump impeller 200 can have different types of blade members. For example, in some embodiments the blade members are inflatable and are inflated while the pump impeller 200 is in its operable configuration. In addition, some such embodiments of the pump impeller 200 include noninflatable web portions attached to such blade members (that are inflated while the pump impeller 200 is in its operable configuration, e.g., see FIG. 12). Each noninflatable web portion can be attached to a respective blade member. Further, in some such embodiments the blade members (that are inflated while the pump impeller 200 is in its operable configuration and that have noninflated web portions attached thereto) can spiral around the axis 201. For example, in some embodiments the blade members can spiral around the axis 201 by at least 30°, or at least 40°, or at least 50°, or at least 60°, or at least 70°, or at least 80°, or at least 90°, or by greater than 90°.

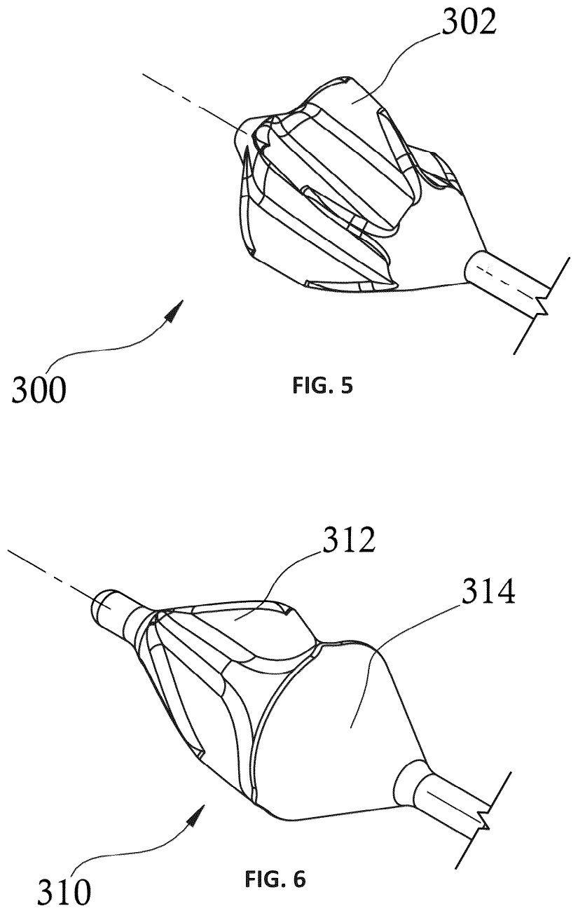

FIGS. 5-9 illustrate various impeller profiles and the surfaces needed to create a hydrodynamic bearing resulting in a self-centering impeller. One or more features of these impeller designs can be combined with one or more design features of other impellers described herein to create hybrid impeller designs, all of which are well within the scope of this disclosure.

FIG. 5 illustrates an impeller 300 that includes ridges 302 that are included to improve the flow dynamics of the impeller 300. The impeller 300 does not have any substantially exposed smooth surfaces to function for the creation of a hydrodynamic bearing. Therefore impeller 300 may not self-center well within a pump housing.

FIG. 6 illustrates an impeller 310 which has ridges 312 to improve the flow dynamics of the impeller 310, as well as an exposed smooth conical surface 314 which may function for the creation of a hydrodynamic bearing. Accordingly, the impeller 310 will tend to self-center within a pump housing during the rotation of the impeller 310.

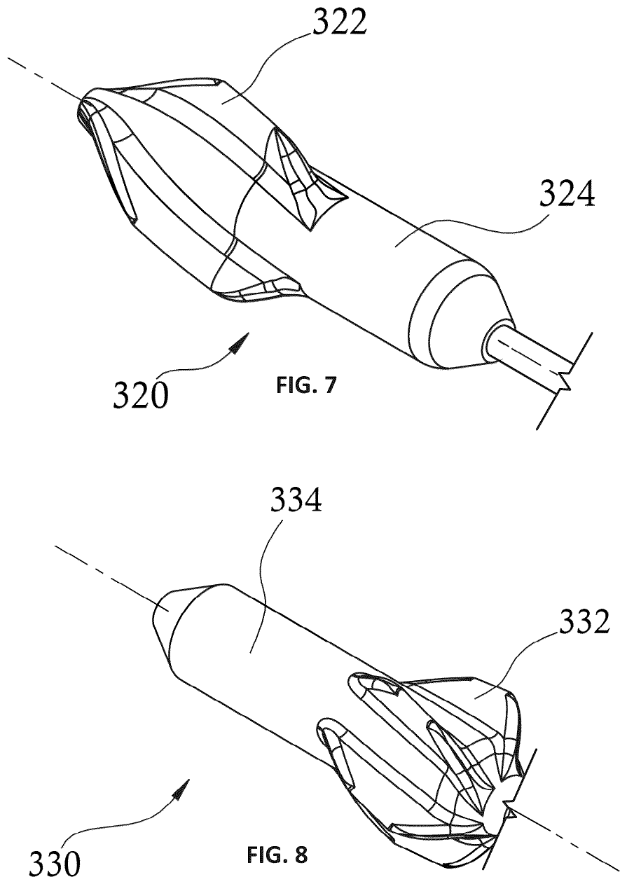

FIG. 7 illustrates an impeller 320 that includes ridges 322 that are similar to the ridges 302 of the impeller 300 (FIG. 5). However, the impeller 320 has the addition of an exposed cylindrical smooth surface 324 protruding proximally from the ridges 322. The cylindrical smooth surface 324 may function to create a robust hydrodynamic bearing that tends to self-center the impeller 320 within a pump housing during the rotation of the impeller 320.

FIG. 8 illustrates an impeller 330 that includes ridges 332 and an exposed cylindrical smooth surface 334 protruding distally from the ridges 332.

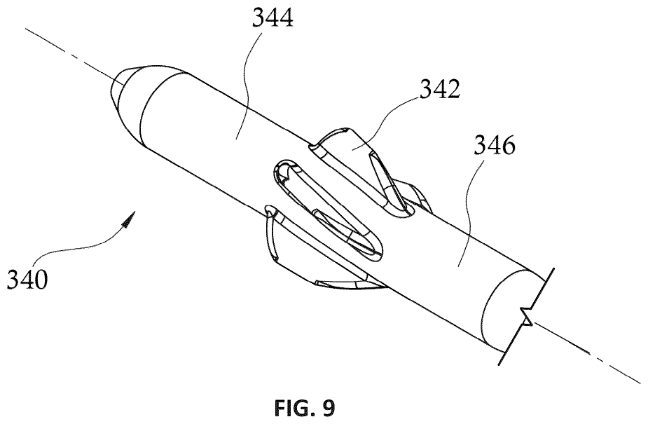

FIG. 9 illustrates an impeller 340 that includes ridges 342 as well as exposed smooth cylindrical surfaces protruding both distally 344 and proximally 346 from the ridges 342, thereby facilitating the creation of a hydrodynamic bearing on both the proximal and distal ends of the impeller 340 for additional self-centering and rotational stability of the impeller 340 during the rotation of the impeller 340 within a pump housing.

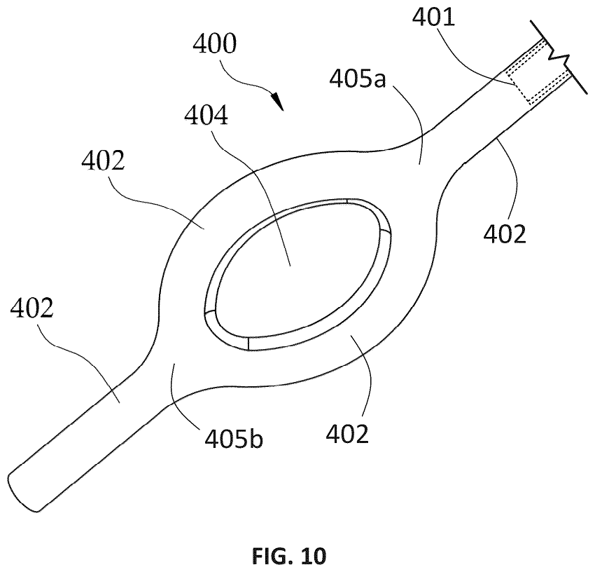

FIGS. 10-12 illustrate some additional example impeller embodiments with inflatable portions and non-inflatable portions. When pressurized, the inflatable portions of these impellers may provide tension along the non-inflatable portions, thereby making them rigid. When the inflatable portions are deflated, the non-inflatable portions may be flexible (to facilitate a low-profile delivery configuration).

FIG. 10 illustrates an impeller 400, that can be attached (e.g., adhered using an adhesive, heat staked, ultrasonic welded, using an interference fit, and the like, and combinations thereof) at a distal end portion of a drive shaft 401. The impeller 400 includes an inflatable portion 402 and a non-inflatable web portion 404 or membrane 404. The drive shaft 401 defines a lumen for conveying inflation medium to and from the inflatable portion 402 to control the pressurization of the inflatable portion 402.

The web portion 404 is attached to and peripherally surrounded by blade members of the inflatable portion 402. In the depicted embodiment, the inflatable portion 402 comprises an oblong torus when inflated (as shown). The web portion 404 is within the oblong torus of the inflatable portion 402.

The inflatable portion 402 comprises a proximal bifurcation 405a from which proximal end portions of a first inflatable blade member and a second inflatable blade member each distally extend. The distal end portions of the first and second inflatable blade members are conjoined at a distal bifurcation 405b of the inflatable portion 402. While in the depicted embodiment of the impeller 400 there are two inflatable blade members, in some embodiments three or more inflatable blade members are included.

When impeller 400 is inflated, the inflatable portion 402 may round out and pull on the membrane 404, thereby maintaining it in a rigid position when the impeller 400 is pressurized (e.g., similar in concept to the spokes of a bicycle tire). When the impeller 400 is not pressurized, the membrane 404 may be flexible and folded into a low-profile delivery configuration.

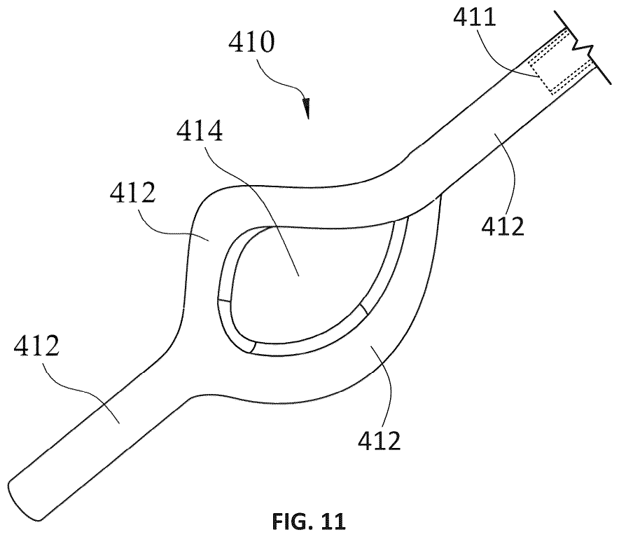

FIG. 11 illustrates an impeller 410 attached (e.g., adhered using an adhesive, heat staked, ultrasonic welded, using an interference fit, and the like, and combinations thereof) at a distal end of a drive shaft 411. The impeller 410 includes an inflatable portion 412 and a non-inflatable web portion 414 or membrane 414. The impeller 410 is similar in construction to the impeller 400 (FIG. 10), however the impeller 410 twists along its central rotational axis which may improve the flow of the impeller 410. Said another way, the inflatable blade members of the inflatable portion 412 extend along spiral paths. While in the depicted embodiment there are two inflatable blade members, in some embodiments three or more inflatable blade members are included that each extend along spiral paths.

When impeller 410 is inflated, the inflatable portion 412 may expand and apply tension on the membrane 414, thereby forming it and maintaining it in a rigid configuration when the impeller 410 is pressurized. When the impeller 410 is not pressurized, the membrane 414 may be flexible and folded into a low-profile delivery configuration.

FIG. 12 illustrates an impeller 420 that can be attached (e.g., adhered using an adhesive, heat staked, ultrasonic welded, using an interference fit, and the like, and combinations thereof) along a distal end portion of a drive shaft 421. The impeller 410 includes an inflatable portion 422. An inflation medium can be supplied to the inflatable portion 422 via a lumen and via one or more fenestrations defined by the drive shaft 421 (at least two such fenestrations are included in the depicted embodiment).

The inflatable portion 422 comprises a first inflatable blade member and a second inflatable blade member. The first and second inflatable blade members extend axially along spiral paths between a proximal bifurcation 425a and a distal bifurcation 425b.

The impeller 420 also includes two non-inflatable membrane or web portions 424. Each non-inflatable web portion 424 is attached to a respective one of the inflatable blade members.

Additionally, the impeller 420 also includes a central portion 426 that extends through and along the axial middle of the impeller 420. The central portion 426 is positioned radially between the inflatable blade members, and axially between the proximal bifurcation 425a and the distal bifurcation 425b.

The distal end portion of the drive shaft 421 extends axially through the central portion 426. Accordingly, the central portion 426 of the impeller 420 can be functionally considered to be a portion of the drive shaft 421 because the central portion 426 adds mechanical structure and rigidity to help establish the physical configuration of the inflated impeller 420 as shown.

The two non-inflatable membrane or web portions 424 are each attached to the central portion 426. Accordingly, each of the non-inflatable membrane or web portions 424 are peripherally bounded by, and attached to, a respective inflatable blade member and the central portion 426 of the impeller 420.

While in the depicted embodiment of the impeller 420 there are two inflatable blade members, in some embodiments three or more inflatable blade members are included that each extend along spiral paths. In some such embodiments, three or more non-inflatable membrane or web portions 424 are also included.

When impeller 420 is inflated, the inflatable portion 422 may round out and pull on the web portions 424, thereby maintaining the web portions 424 in rigid configurations when the impeller 420 is pressurized. When the impeller 420 is not pressurized, the web portions 424 may be flexible and folded into a low-profile delivery configuration.

Impellers 400, 410, and 420 (all described above) illustrate example impellers which, in some cases, may be pressure inflated instead of volumetrically inflated, and maintain the desired operable geometry. Furthermore, the inflation pressure used to expand the impeller may provide further structural and geometric stability to ensure the impeller does not flex under the external forces exerted on the impeller when rotating at high speeds within a fluid.

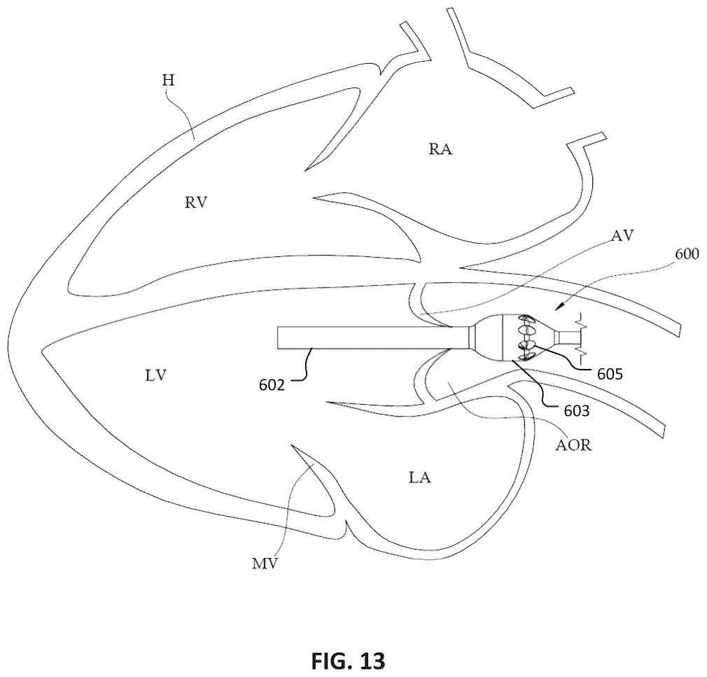

FIG. 13 illustrates an expanded and operational pVAD 600 (in accordance with the pVAD pump impellers and pVAD systems described herein) placed across the aortic valve (“AV”) with the inlet cannula 602 placed in the left ventricle (“LV”) and the pump housing 603 located in the aortic root (“AOR”) of the aorta. The wall openings 605 of the pump housing 603 allow blood that is pumped by the pVAD 600 to exit into the aorta from the pump housing 603. This placement of the pVAD 600 is an example operational placement that facilitates the pumping of blood from the LV to the rest of the body using the pVAD 600.

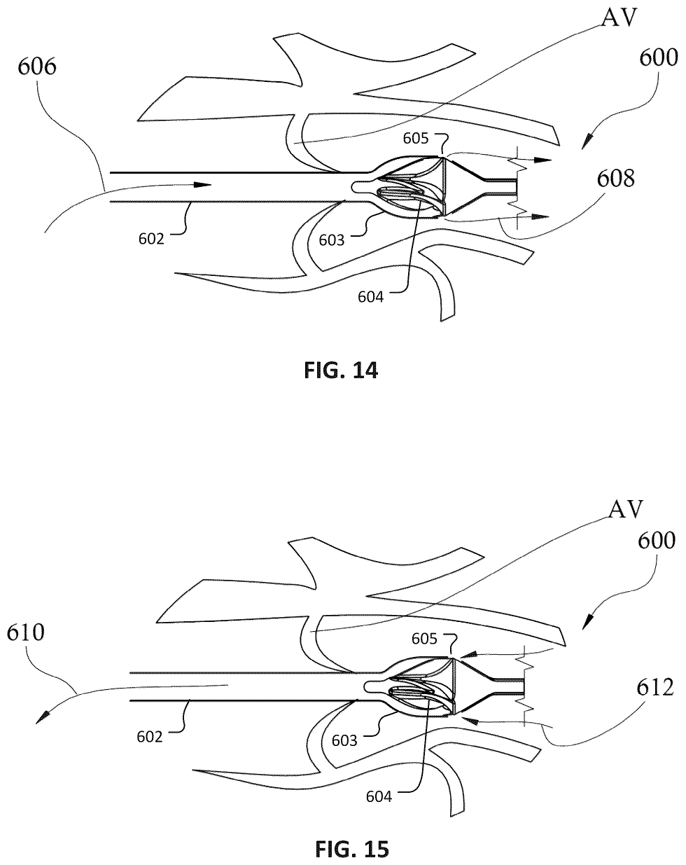

Referring also to FIG. 14, here the pVAD 600 is illustrated in a cross-sectional view that reveals a pump impeller 604 within the pump housing 603. In FIG. 14, the pump impeller 604 is located at its proximal-most location within the pump housing 603. This is an operational location of the pump impeller 604 within the pump housing 603. Accordingly, when the pump impeller 604 is rotated, blood flow 606 enters the inlet cannula 602 from the LV, and blood flow 608 exits the wall openings 605 of the pump housing 603 in the aorta.

FIG. 15 illustrates the same cross-sectional view of the expanded pVAD 600 as FIG. 14. However, FIG. 15 illustrates that retrograde blood flow can occur if the pump impeller 604 stops rotating while the pump impeller 604 is located at its proximal-most location within the pump housing 603. That is, if the pump impeller 604 stops rotating for whatever reason, the pressure gradient across the AV (e.g., during diastole) may cause blood flow 612 from the aorta to enter the pump housing 603 through the wall openings 605 and blood flow 610 to exit the inlet cannula 602 in the LV. When blood flows in a backwards direction (retrograde) through the AV during diastole (as in this example), it is a cause of aortic insufficiency that can have negative repercussions for the patient.

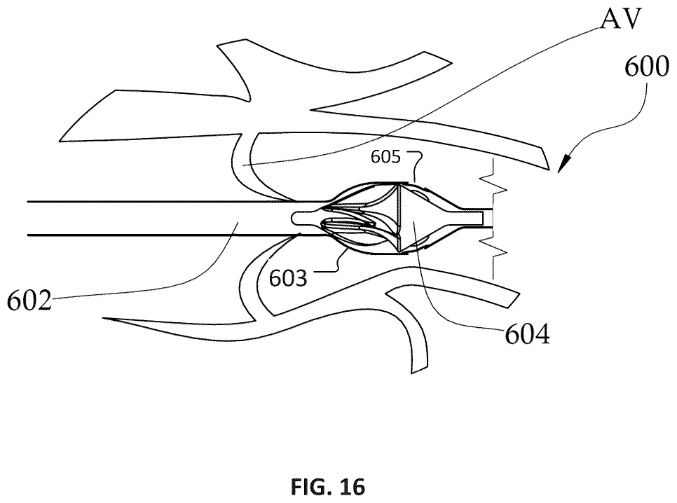

FIG. 16 illustrates a method for remedial mitigation or prevention of the retrograde blood flow scenario illustrated in FIG. 15. That is, when the pump impeller 604 stops rotating, the pump impeller 604 can be pushed distally to position it farther distally within the pump housing 603. With the pump impeller 604 in this distal position (as shown in FIG. 16), the pump impeller 604 may act as a plug to reduce or prevent blood from the aorta from flowing into the pump housing 603 through its wall openings 605. While this would not be desired during normal operation of the pVAD 600, if the impeller 604 were to unexpectedly stop spinning, advancing the impeller 604 farther distally within the pump housing 603 could reduce the potential of aortic insufficiency resulting from retrograde blood flow through the pVAD 600, thereby reducing the negative effects on the patient.

### Optional Features and Additional Embodiments

Some embodiments of the inflatable impellers include blade members that extend spirally around the central longitudinal axis of the impeller. The extent of the spirals can be different depending on the desired pumping characteristics of the impeller. In some embodiments, the blade members can spiral by at least 30°, or at least 60°, or at least 90°, or at least 120°, or at least 150°, or at least 180°.

With general reference to inflatable balloon-based pump impellers, some embodiments may utilize the fact that the impeller spins at a high rpm to assist with impeller expansion. That is, when the pump impeller is spinning, it imparts centrifugal force on the inflation medium within the impeller. The impeller will tend to expand in response to the inflation medium's centrifugal force. As a result, additional inflation medium will be drawn into the impeller to fully expand the impeller. The centrifugal force may be sufficient to maintain a balloon in an inflated or expanded operational state. When centrifugal force is used in such a manner, then the rotatable inner catheter need not be a fully sealed system in some examples.

An inflatable balloon-based pump impeller may be inflated, for example, via a luer adaptor using standard balloon catheter inflation medium (e.g., a mixture of saline and contrast), or it may also be inflated with a more viscous fluid or a higher density fluid. The impeller may also be inflated with a fluid that takes a gelatinous set to help the impeller maintain its surface features once expanded and spinning. To deflate the impeller that is filled with a liquid that sets may require the additional delivery of a chemical or enzyme to break down the gelatinous structure. If the impeller is inflated with a mixture of saline and contrast, for example, then it may be deflated with standard balloon deflation techniques (e.g., suction of the inflation medium).

In some embodiments, the impeller may have one or a plurality of radiopaque markers that help the user identify the location of the impeller in relation to the patient anatomy and in relation to the pump housing to ensure correct placement. These radiopaque markers may be placed either on the impeller membrane, or on the drive shaft within the impeller, or a combination of both locations. Additionally, in some embodiments the inflation medium used to expand the impeller may be radiopaque, thereby allowing the full impeller to be visible under fluoroscopy.

With general reference to the catheter-based blood pumps described herein, there are many varying use scenarios where a blood pump may support a patient's vitals and improve their outcome, in accordance with embodiments of the subject matter disclosed herein. The catheter-based blood pump will be used to provide mechanical circulatory support to patients who may need additional blood pumping capabilities and/or capacities. This may be used either as a long-term ventricular assist device or as a short-term device to perform protected PCI but is not restricted to these use scenarios. The invention can gain patient access either through a conventional femoral access site or through an upper body access site, for example a radial access site. Using a radial access site may allow a patient to be ambulatory with the device still in place and pumping blood. A radial access may also allow for the femoral arteries to be used for other concomitant procedures, such as but not limited to percutaneous angioplasty/stenting and lower limb revascularization.

Many alternate access sites may be used for placing the apparatus because a large bore access site is not required for placing the device within a patient. In addition to a radial or femoral artery access site, the access site may be in the femoral veins or in the brachial vessels. The axillary arteries may also be used as access sites for placement of the device. Additionally, in a blood pushing configuration, the device may be placed through a trans-thoracic/trans-apical approach and pump blood from the left ventricle into the aorta.

The apparatus may be placed across the aortic valve and pump blood from the left ventricle into the aorta as illustrated in FIG. 1. In a blood pushing configuration the apparatus may be placed in the right ventricle and across the semi-lunar valve to pump blood into the pulmonary artery. A blood pushing configuration may also be placed in the left ventricle through a trans-apical or trans-septal access pathway.

To provide general context, Table 1 (below) lists some example size ranges of various components of some embodiments of the percutaneous ventricular assist devices described herein. It should be understood, however, that these are non-limiting examples, and that the components are scalable to various other desired sizes and size ranges, both smaller and larger than listed in Table 1.

TABLE 1

| | Collapsed | | | | |
|---|---|---|---|---|---|
| | Inlet | | | | |
| Introducer | Cannula | Expanded | Expanded | | |
| Sheath | and Pump | Inlet | Pump | Collapsed | Expanded |
| Compati- | Housing | Cannula | Housing | Impeller | Impeller |
| bility | Diameters | Diameters | Diameters | Diameters | Diameter |
| ≤6Fr | <.078″ | 9Fr-21Fr | 14Fr-30Fr | <.071″ | 9Fr-27Fr |
| 7Fr | .078-.090″ | 12Fr-24Fr | 24Fr-42Fr | <.078″ | 18Fr-39Fr |
| ≥8Fr | >.090″ | 12Fr-30Fr | 24Fr-78Fr | >.078″ | 21Fr-75Fr |

The percutaneous ventricular assist devices described herein may be operated at various speeds (e.g., in terms of revolutions per minute (“rpm”) of the pump impeller) in order to obtain the desired operational pumping rate performance. In some non-limiting example embodiments, the pump impeller of the percutaneous ventricular assist devices described herein may be operated in a range of 0 rpm to 10,000 rpm, or 5,000 rpm to 20,000 rpm, or 5,000 rpm to 30,000 rpm, or 5,000 rpm to 40,000 rpm, or 5,000 rpm to 50,000 rpm, or 10,000 rpm to 20,000 rpm, or 10,000 rpm to 30,000 rpm, or 10,000 rpm to 40,000 rpm, or 10,000 rpm to 50,000 rpm, or 10,000 rpm to 60,000 rpm, or 20,000 rpm to 30,000 rpm, or 20,000 rpm to 40,000 rpm, or 20,000 rpm to 50,000 rpm, or 20,000 rpm to 60,000 rpm, or 30,000 rpm to 40,000 rpm, or 30,000 rpm to 50,000 rpm, or 30,000 rpm to 60,000 rpm, or 40,000 rpm to 50,000 rpm, or 40,000 rpm to 60,000 rpm, or 50,000 rpm to 60,000 rpm, or greater than 60,000 rpm. It should be understood that these ranges of pump impeller rpms are purely exemplary and non-limiting as the actual impeller rpm used during a procedure will ultimately depend on many different factors such as, but not limited to, impeller design, pump system size, patient parameters, clinician preferences, and so on.

The devices described herein may be optionally MRI compatible. The drive shaft, and drive shaft housing may be made from an MRI safe support structure material, such as nitinol or PEEK etc., and the sheath, impeller and casing may also be MRI compatible, so the full implantable portion of the apparatus would be MRI compatible. Therefore, with an extended length drive shaft and drive shaft housing outside of the body it would be possible to provide patients with hemodynamic support during MRI imaging.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. A medical pump comprising:
an elongate drive shaft defining a lumen; and
an impeller attached to a distal end of the drive shaft and comprising:
an inflatable portion fluidly coupled to the lumen; and
a noninflatable web portion attached to and peripherally surrounded by the inflatable portion.

2. The medical pump of claim 1, wherein the inflatable portion comprises an oblong torus when inflated, and wherein the web portion is within the oblong torus.

3. The medical pump of claim 1, wherein the inflatable portion comprises a first inflatable blade member extending from the distal end of the drive shaft and a second inflatable blade member extending from the distal end of the drive shaft, and wherein distal end portions of the first and second inflatable blade members are conjoined at a distal end of the inflatable portion.

4. The medical pump of claim 3, wherein the first and second inflatable blade members extend along spiral paths from the distal end of the drive shaft to the distal end of the inflatable portion.

5. The medical pump of claim 1, wherein the inflatable portion comprises three or more inflatable blade members that each extend from the distal end of the drive shaft and that conjoin at a distal end of the inflatable portion.

6. The medical pump of claim 5, wherein the three or more inflatable blade members extend along spiral paths from the distal end of the drive shaft to the distal end of the inflatable portion.

7. A medical pump comprising:
an elongate drive shaft defining a lumen; and
an impeller attached to a distal end portion of the drive shaft and comprising:
a first inflatable blade member extending spirally between a proximal end and a distal end of the first inflatable blade member, wherein the proximal and distal ends of the first inflatable blade member are each attached to the distal end portion of the drive shaft;
a second inflatable blade member extending spirally between a proximal end and a distal end of the second inflatable blade member, wherein the proximal end distal ends of the second inflatable blade member are each attached to the distal end portion of the drive shaft;
a first noninflatable web portion attached to the first inflatable blade member and to the distal end portion of the drive shaft; and
a second noninflatable web portion attached to the second inflatable blade member and to the distal end portion of the drive shaft.

8. The medical pump of claim 7, wherein the impeller further comprises:
a third inflatable blade member extending spirally between a proximal end and a distal end of the third inflatable blade member, wherein the proximal end distal ends of the third inflatable blade member are each attached to the distal end portion of the drive shaft; and
a third noninflatable web portion attached to the third inflatable blade member and to the distal end portion of the drive shaft.

9. The medical pump of claim 7, wherein the impeller is reconfigurable between a deflated low-profile delivery configuration and an inflated operable configuration that is radially expanded in comparison to the low-profile delivery configuration.

10. The medical pump of claim 9, wherein, when the impeller is in the inflated operable configuration, the first and second impeller blades each spiral around the distal end portion of the drive shaft by at least 120°.

11. The medical pump of claim 9, wherein, when the impeller is in the inflated operable configuration, the first and second impeller blades each spiral around the distal end portion of the drive shaft by at least 180°.

12. A medical pump comprising:
an inflatable impeller that is rotatable about an axis and that has an operable configuration comprising:
an inflated conical portion; and
multiple blade members extending axially from the conical portion.

13. The medical pump of claim 12, wherein the multiple blade members are inflatable, and wherein the multiple blade members are deflated while the inflatable impeller is in the operable configuration.

14. The medical pump of claim 12, wherein the multiple blade members consist of three blade members.

15. The medical pump of claim 12, further comprising an elongate drive shaft, and wherein the conical portion is attached to a distal end portion of the elongate drive shaft.

16. The medical pump of claim 12, wherein the inflatable impeller is configurable in three configurations comprising:
the operable configuration;
an uninflated low-profile delivery configuration; and
a fully inflated configuration,
wherein the operable configuration includes the multiple blade members but the fully inflated configuration does not include the multiple blade members.

17. The medical pump of claim 12, wherein the multiple blade members are inflatable, wherein the multiple blade members are inflated while the inflatable impeller is in the operable configuration, wherein the inflatable impeller further comprises a plurality of noninflatable web portions, wherein each noninflatable web portion is attached to a respective blade member of the multiple blade members.

18. The medical pump of claim 17, wherein, when the inflatable impeller is in the operable configuration, the multiple blade members each spiral around the axis by at least 50°.

19. The medical pump of claim 12, wherein the multiple blade members comprises three blade members that are each inflatable, wherein the inflatable impeller further comprises three noninflatable web portions, and wherein each noninflatable web portion of the three noninflatable web portions is attached to and located radially within a respective blade member of the three blade members.

20. The medical pump of claim 19, wherein, when the inflatable impeller is in the operable configuration, the three blade members each spiral around the axis by at least 50°.
